(19) United States
(12) Patent Application Publication
(10) Pub. No.: US 2017/0004266 A1
(43) Pub. Date: Jan. 5, 2017

(54) METHOD OF ADMINISTERING A HEALTH CARE CODE REPORTING SYSTEM

(71) Applicant: Gremlo LLC, St. Louis, MO (US)

(72) Inventor: Eric Vanderhoef, St. Louis, MO (US)

(21) Appl. No.: 15/201,109

(22) Filed: Jul. 1, 2016

Related U.S. Application Data
(60) Provisional application No. 62/187,347, filed on Jul. 1, 2015.

(51) Int. Cl. G06F 19/00; G06F 17/30

(52) U.S. Cl. CPC G06F 19/328; G06F 17/30867; G06F 19/322; G06F 17/30554; G06F 17/30371

(57) ABSTRACT

A method of administering a healthcare analytics process through a computer system having at least one server, at least one client device, and a communication network operatively and electrically connecting the client device to the at least one server, the method comprising the steps of: providing a coding program running on the at least one server without transmitting advertisements to the at least one client device; accessing, by a user from the client device, the coding program, and entering search data into the at least one client device by the user; transmitting the search data to the at least one server; and generating with the coding program on the at least one server, search results associated with the search data and displaying the search results associated with the search data at the at least one client device.

### CROSS-REFERENCE TO RELATED APPLICATIONS

This non-provisional application claims priority to U.S. Provisional Application Ser. No. 62/187,347, filed Jul. 1, 2015, and which is incorporated herein by reference.

### STATEMENT REGARDING FEDERALLY SPONSORED RESEARCH

Not applicable.

### BACKGROUND

The medical billing process is an interaction between a health care Provider (identified by medical doctors, doctors of osteopathic medicine, physician assistants and nurse practitioners) and the payer, such as an insurance company or The Centers for Medicare & Medicare Services (CMS). The entirety of this interaction is known as the billing cycle. This can take anywhere from several days to several months to complete, and require several interactions before a resolution is reached.

Each bill contains a Current Procedural Terminology (CPT) Code (work unit provided by the Provider) and an International Statistical Classification of Diseases (ICD) code to describe the medical condition(s) experienced by the patient causing them to seek medical care from a Provider (or at the request of the Provider as part of the management of a wellness condition). In order to receive payment of a medical billing claim, the Provider or medical biller must have all the data elements required in an electronic claim, complete knowledge of different insurance plans and the laws, and regulations that preside over them. Medicare Advantage Plans, Managed Medicaid, Accountable Care Organizations, Commercial Health insurance, Government Health Exchanges, Tricare and Self-funded insurance plans all require claims to be submitted electronically.

Quality metrics are also increasingly important to the measurement of the healthcare system. The push for appropriate intervention and management of key disease states by Providers are the new norm. Programs such as CMS's Medicare STARS (assessing quality for Medicare Advantage Plans), Bundled Payments (flat rates for all healthcare intervention for a particular disease state, currently orthopedic events such as hip replacements and knee replacements are being piloted), Fee for value where Providers are paid on both the work units as well as the outcomes linked many times to the patients perception of quality as it relates to their Provider's clinical acumen are some of the many new programs coming out at the time of the filing. The key pieces for success under this new paradigm are actionable and comprehensive information available at the point of care.

CMS, Employers (entities providing health insurance to their employees) and private insurance companies (including Commercial, Medicare, Medicaid, self-insured and Tricare) are attempting to reduce the total spend on healthcare. Numerous programs such as the STAR Ratings (quality measurement for Medicare Advantage) or annual wellness screenings (provision in the Patient Protection Affordable Care Act) are attempting to bend the cost curve through mandate. The challenge is that Providers lack timely, actionable information at the point of care to assist in diagnosing and managing wellness conditions present in their patients.

Providers have been resistant to external healthcare analytics because they violate three key cannons of the modern Provider practice: 1) do not ask Providers to do outside research on their patients; 2) do not ask Providers to enter any data into a software; and 3) do not attempt to modify the patient flow in their offices. Gremlo's solution works within these metrics and still creates actionable information that is easily adopted by the Provider in the management of their patients.

CMS reimburses Medicare Advantage health plans based on the health status of the enrolled member, referred to as “The Risk Adjustment Factors” (RAF). CMS uses claims data captured through claims data submitted by Medicare Advantage Plans, Managed Medicaid and Accountable Care Organizations (ACOs) to group patients into risk adjustment categories and assign patient specific payments to the health plans based on a member's health status. Data comes from claims submitted by Providers to health plans based on the diagnosis and treatment of patients by Providers. Specifically, CMS determines the risk for each member based on the diagnostic codes, such as (ICD-10), entered from the medical record. Currently, the CMS mandates that the diagnostic codes comply with the International Statistical Classification of Diseases, Tenth Revision (ICD-10). Some of the diagnostic codes are assigned a corresponding risk factor score or Hierarchical Condition Category (HCC). Under this system, Providers can generate additional payments for members with certain medical conditions. Therefore, for a Medicare Subcontractor such as Medicare Advantage health plan, Accountable Care Organization (ACO), Managed Medicaid health plan or an entity accepting risk from CMS for the management of an individual's health status. To receive the full payment from CMS for the management of each patient (or in health plans case the member) for the health plan it requires Providers to accurately diagnose and capture in their medical charts and on their claims valid diagnosis codes (ICD-10). The onus is on the Provider to generate complete and accurate assessments in their medical records and on their claims. Incomplete or inaccurate data will impact the revenue paid by CMS to the health plans. Providers that do not accept risk from CMS are paid according to CPT codes, but are still required to submit at least one ICD-10 code for their medical claim to be paid. The lack of Provider focus on accurate diagnosing, charting and coding of all the present conditions at the point of care is a key piece to the importance of this patent being filed. Active management is increasingly important for Providers in fee for service medicine as CMS is pushing aggressively for Providers to be paid through capitation, bundled payments or Value Based Purchasing (aka. Global risk).

The complexity of selecting and entering the appropriate codes from the array of 64,000 ICD-10 codes can result in errors. In fact, coding is so complicated that the individuals that enter codes require specialized training and certification. In addition, Providers are paid on CPT Codes, not ICD-10 Codes. The diagnoses codes (ICD-10) are rarely coded and managed by Providers to the highest degree of specificity recommended for the wellness conditions present in the patient. A claim requires only one condition (ICD Code) that corresponds to a valid CPT Code (work unit Providers are paid in fee for service medicine) for a claim to be paid. This leads to less reported diagnoses and ICD Codes then are present and a strong indication of a lack of Provider Management of all the wellness conditions in the Provider's Patients, key component to population healthcare or the holistic management of the patient.

The process of accurate and specific diagnosing (ICD Codes) and management of conditions by the Provider at the point of care has numerous other important applications. In addition to aforementioned health plans, other applications of information from the Provider-patient encounter can include, but are not limited to:
Employers who self-fund their insurance: Use for creating wellness intervention points with their employees. The goal of population healthcare or holistic management of wellness conditions is to increase compliance by patients with Provider's advice and decrease avoidable manifestations of chronic conditions (avoidable healthcare costs) to an expensive episode of care.
Designing disease management/case management/utilization management programs. By clearly identifying the “at risk” people for a costly episode of care, specific interventions from Providers can help mitigate the risk. By helping Providers more effectively manage wellness conditions in their patients with actionable information, many chronic conditions care be more effectively managed in the lower cost Provider Office. Left unmanaged, chronic conditions can manifest into an avoidable high cost episode of care.
Other key government sponsored health insurance: Programs such as the Affordable Care Act's Health Exchanges and the Department of Defense's (DOD) Tricare and Veteran's Affairs insurance options. These entities are seeking intervention points to reduce the costs of care. Actionable clinical information is important for Providers to increase utilization of Provider's appointment times and information to coordinate between private Providers and the base military health systems to reduce healthcare spend for the DOD.
Provider process improvement: The ability to measure and monitor how a Provider practices medicine in totality gives key indications for process improvement in holistic patient management. By using this information to show how a Provider is managing all the wellness conditions present in their patients (population healthcare) Providers can improve their own methods for managing their patients in their individual practice of medicine. These changes will become even more important as this information is one of the key success factors in Provider's success under Health and Human Services and health insurance companies push Providers to move from fee for service medicine to Providers accepting risk contracts for Value Based Purchasing and Capitation.

Therefore, there is a long felt need for a method and apparatus that allows Providers identify and enter the correct codes to receive full payment of medical billings.

### DESCRIPTION OF THE DRAWINGS

In the accompanying drawings which form part of the specification:

FIG. 1 is a block diagram of a system for administrating a coding process in accordance with the present invention;

FIG. 2 is a sample homepage webpage appearing on the display of the client device and displaying search results;

FIG. 3 is a sample input form webpage appearing on the display of the client device and displaying search results;

FIG. 4 is a sample coding results webpage appearing on the display of the client device;

FIG. 5 is a sample report appearing on the display of the client device;

FIG. 6 is a sample homepage webpage appearing on the display of the client device and displaying search results.

Corresponding reference numerals indicate corresponding parts throughout the several figures of the drawings.

### DETAILED DESCRIPTION

The following detailed description illustrates the claimed invention by way of example and not by way of limitation. The description clearly enables one skilled in the art to make and use the disclosure, describes several embodiments, adaptations, variations, alternatives, and uses of the disclosure, including what is presently believed to be the best mode of carrying out the claimed invention. Additionally, it is to be understood that the disclosure is not limited in its application to the details of construction and the arrangements of components set forth in the following description or illustrated in the drawings. The disclosure is capable of other embodiments and of being practiced or being carried out in various ways. Also, it is to be understood that the phraseology and terminology used herein is for the purpose of description and should not be regarded as limiting.

Medical billing begins with the office visit: a doctor or their staff will typically create or update the patient's medical record (Predominantly Electronic Medical Records due to Meaningful Use guidelines from CMS). This record contains a summary of treatment and demographic information including, but not limited to, the patient's name, address, social security number, home telephone number, work telephone number and their insurance policy identity number. If the patient is a minor, then guarantor information of a parent or an adult related to the patient will be appended. Upon the first visit, the Provider will usually give the patient one or more diagnoses in order to better coordinate and streamline their care. In the absence of a definitive diagnosis, the reason for the visit will be cited for the purpose of claims filing. The patient record contains highly personal information, including the nature of the illness, examination details, medication lists, diagnoses, and suggested treatment.

The extent of the physical examination, the complexity of the medical decision making and the background information (history) obtained from the patient are evaluated to determine the correct level of service that will be executed by the Provider and billed to the insurance company.

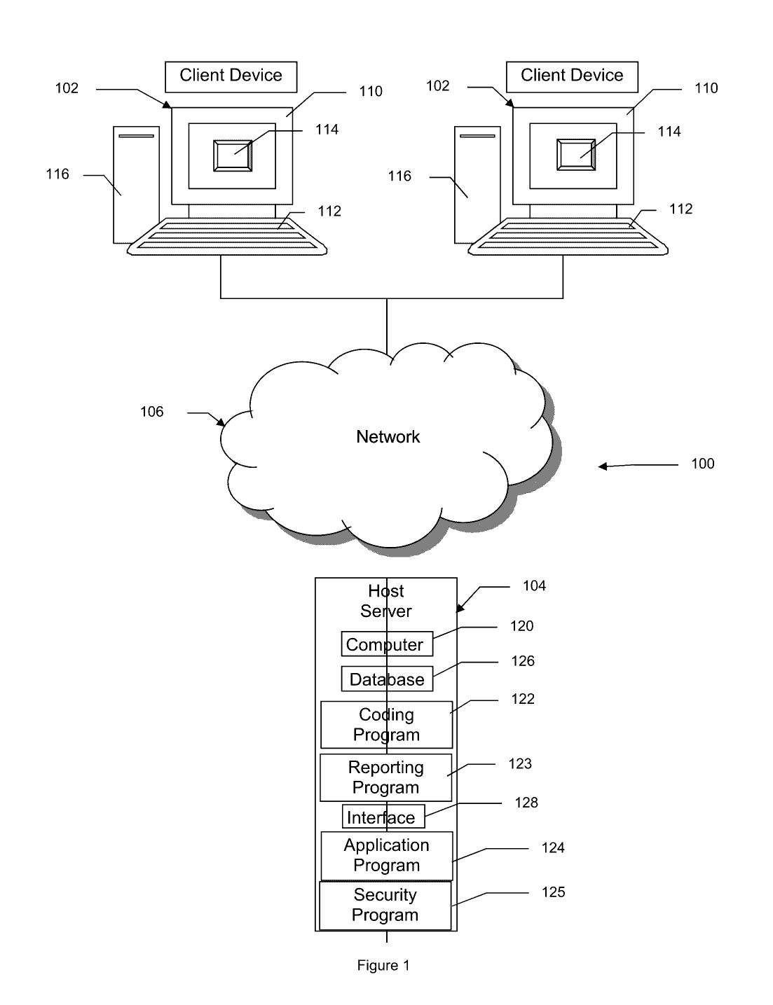

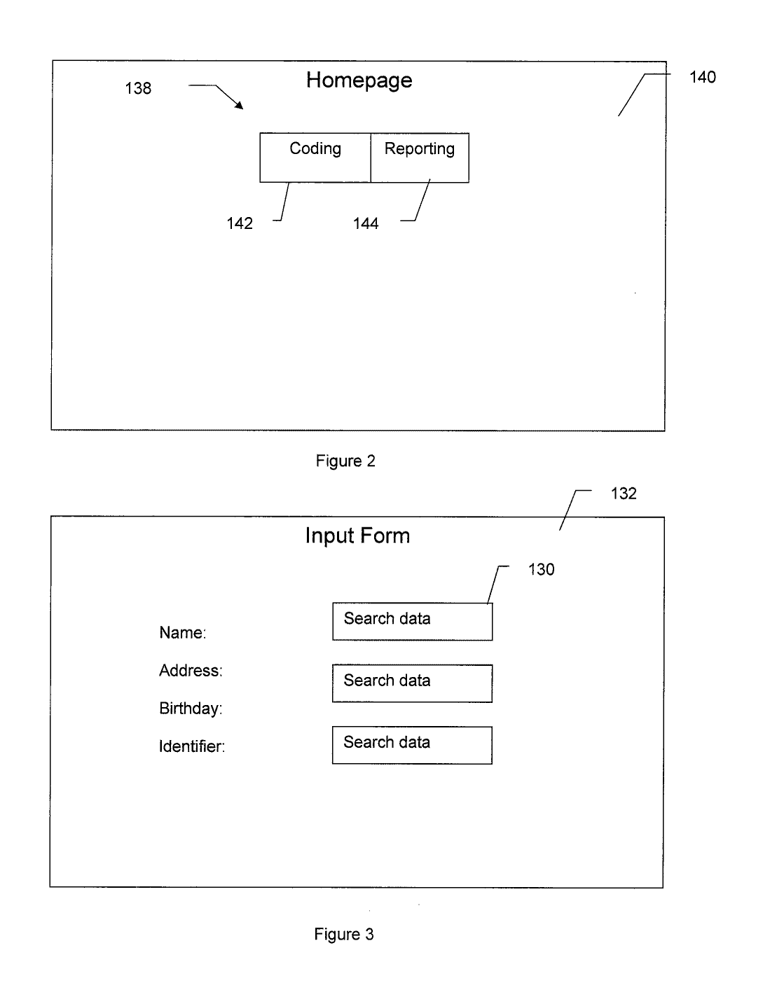

As shown in FIG. 1, an embodiment of the present invention, generally referred to as a computer system 100, includes at least one client device 102 operatively connected to at least one host server 104 through a communication network 106 to communicate data between the client device 102 and the host server 104. The computer system 100 is capable of administering a coding program 122 and reporting program 123, which is described below in further detail.

In the embodiment of FIG. 1, the client device 102 is a computer 108, including a processor, memory, a mass storage device, a display device 110, and an input device 112, such as a keyboard, that is capable of running a network interfacing program 114, such as web browser software available, for example, from Netscape® Corporation, Apple® Corporation, or from Microsoft® Corporation. The client device 102 is appropriately equipped with a network interfacing device 116 for communicating data with the network 106, such as a dial-up modem, a cable modem, a satellite connection, a DSL (Digital Subscriber Line) connection, a LAN (Local Area Network), or the like. Alternate embodiments of client device 102 include any electrical or electronic device capable of communicating with the server 104 through the network 106, such as, for example, a personal digital assistant (PDA), cellular phone, a telephone operating with an interactive voice-system, or a television operating with a cable or satellite television interactive system.

A user interacts with the client device 102 by viewing data via the display 110 and entering data via the keyboard 112, or other suitable input interface such as a mouse, microphone, touch screen, and the like. The network interfacing program 114 allows the user to enter addresses of specific web pages to be retrieved, which are referred to as Uniform Resource Locators, or URLs. The web pages can contain various types of content from plain textual information to more complex multimedia and interactive content, such as software programs, graphics, audio signals, videos, and so forth. A set of interconnected web pages, usually including a homepage, are managed on a server device as a collection collectively referred to as a website. The content and operation of such websites are managed by the server device, such as host server 104, which is operatively connected to the network 106.

In the embodiment of FIG. 1, the network 106 is the Internet, which uses a suitable communications protocol, such as HyperText Transfer Protocol (HTTP), to communicate data between the client devices 102 and the host server 104. However, the network 106 can be any network that allows an exchange of data between the client devices 102 and the host server 104, such as a LAN or WAN (Wide Area Network). In addition, any suitable type of communications protocol can be used, such as FTP (File Transfer Protocol), SNMP (Simple Network Management Protocol), TELNET (Telephone Network), and the like.

The host server 104 preferably comprises a computer system 120, having a processor, memory, and a mass storage device, which is capable of running a coding program 122 and reporting program 123. A database 126 is stored on the mass storage device. Also, the host server 104 is appropriately equipped with a network interfacing device 128 for communicating data with the network 106, such as a dial-up modem, a cable modem, a satellite connection, a DSL connection, a LAN, or the like. If necessary to accommodate large amounts of information or run numerous applications, alternate embodiments of the host server 104 can comprise multiple computer systems, multiple databases, or any combination thereof. The host server 104 also preferably includes a security program 125 to protect the storage and transfer of all electronic information.

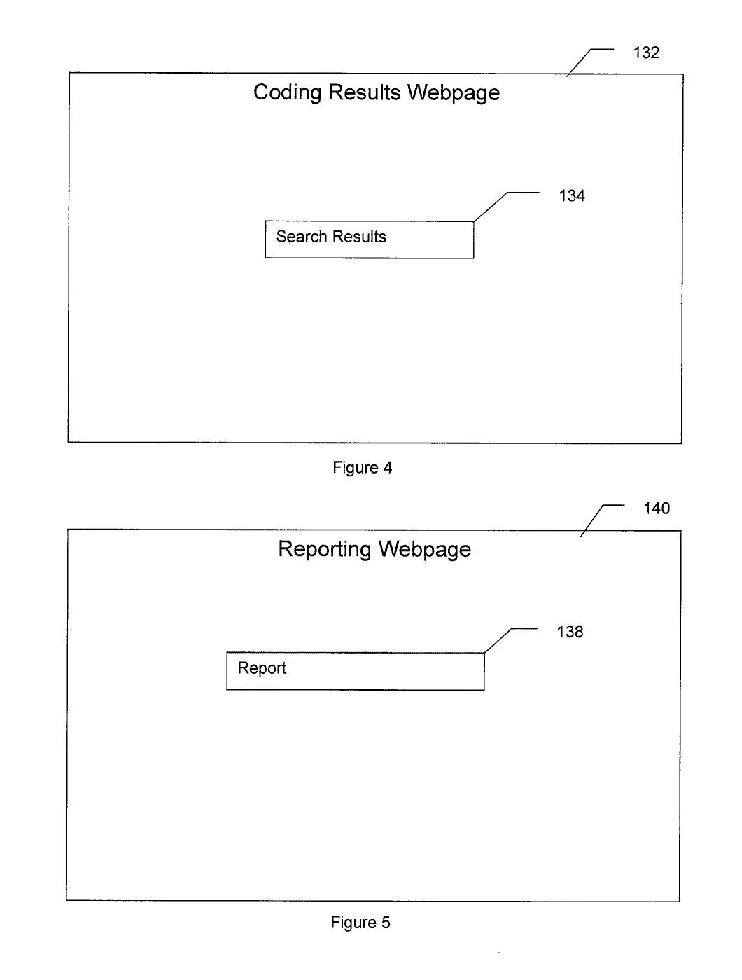

An application program 124 allows users through the interfacing program 114 of the client device 102 to access various service programs 138 on the host server 104. As shown in FIGS. 1 and 4, the application program 124 generates a web page, such as home page 140, that transmits through the network 106 and displays on the client device display 110 via the interfacing program 114. In the preferred embodiment, the home page 140 includes a menu of the various service programs 138 including Coding 142 and Reporting 144. Preferably, the coding program 122 and reporting program 123 operate independently. The features of the coding program 122 should be available to physicians and coders for identification of ICD-10 codes. The features of the reporting program 123 should be available to physicians and designated proxies for identification of clinical indicators that may be relevant to the care of the patients. The user, such as a coder or physician, interacts with the application program 124 by entering data with the input device 112, in this case by selecting one of the service programs 138. By selecting one of these options, the application program 124 generates additional web pages and interacts with the database 126 and the client device 102 in order to provide the selected service programs 138 to the user.

The coding program 122 allows users through the interfacing program 114 of the client device 102 to submit queries to identify data, such as diagnosis codes or HCC's, by entering search data 130, such as medical records, into a web page 132 as shown in FIG. 3. The search data 130 can include, but is not limited to, patient name, patient address, patient birthday, a unique identifier, and patient history. In addition, the search data 130 includes at least in part data that is determined or required by regulatory requirements, which can periodically change. Once entered, the information can be stored on the database 126 of the host server 104. Subsequent queries to identify data can locate the data stored on the database 126 and eliminate the need to reenter unchanged data.

Data can be processed through the system 100 electronically from multiple sources, such as, claims, electronic medical records, government data files from CMS, or health plan raw data files.

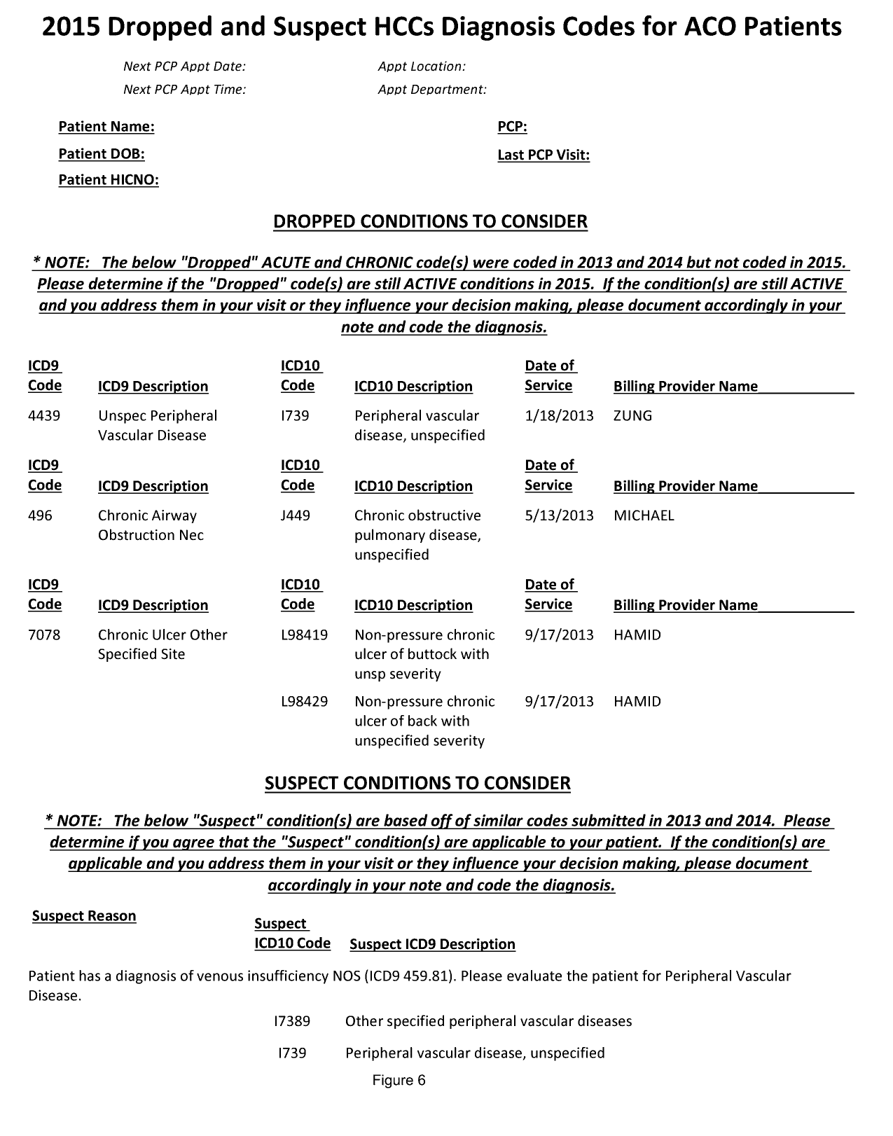

Based on the search data 130, the coding program 122 generates search results 134 from an index of search records on the database 126. The search results 134 include, at least in part, a list of diagnosis codes, such as ICD-10, and HCC's relevant to the search data 130. The coding program 122 uses algorithms to identify data, including, but not limited to; how a Provider is practicing medicine; description of historic patient interventions; suspect logic for Providers to consider based on clinical algorithms; specificity of diagnoses; disease intervention opportunities; plus, appropriate intervention opportunities on HCC's to appropriately capture CMS's payment to subcontractors, such as Medicare Part C Plans, ACO's, and Medicaid companies. The host server 104 transmits the search results 134, preferably in an electronic format such as, a webpage, PDF, or Excel spreadsheet 136 as shown in FIG. 4, to the client device 102, where it is displayed on the display 110. Those skilled in the art will recognize that any typical search engine program, such as Google™, Yahoo!®, MSN®, Ask.com™, and the like, can be used. In this way, the coding program 122 increases the accuracy of coding and reduces the number of rejected claims. FIG. 6 shows a sample page of search results 134 produced by the algorithms of the coding program. The search results may include, but are not limited to:

A centralized combined document for all medical conditions from inpatient, outpatient, physician and pharmacy data bases;

Identification of all ICD-9 codes used from different Providers to describe wellness conditions for the same patient;

A conversion from ICD9 to ICD-10, including both direct crosswalks as well as array of codes for the 70% of the ICD-10 codes that require additional specificity in the diagnosis for the Provider. Note: ICD-10 has 64,000 codes where ICD-9 only had 13,000;

The last date of service the condition was addressed. This is a starting point for Provider to validate diagnosis is still present and to develop treatment plan for conditions if warranted;

The Provider that diagnosed the condition. This provides opportunity for collaboration between physicians on conditions identified; or

Any suspect diagnosis. The system 100 uses previous condition diagnosis and applies best practice Provider methodology for diagnosing and documentation of patient conditions. The system 100 does not replace the physician judgement, but rather gives clinical suggestions on conditions for Providers to consider when diagnosing all the wellness conditions present in their patients. The algorithms are configured to address paired disease codes (for example diabetes, renal disease and chronic kidney disease are frequently found at the same time) and areas where increased specificity of Provider diagnoses could lead to a different patient management regiment from the Provider.

The reporting program 123 allows users through the interfacing program 114 of the client device 102 to submit queries to generate reports based on previously entered search data 130, such as medical records, stored on the database as shown in FIG. 5. The reporting program 123 can generate reports about, for example, members without visits, ICD-10's not coded in the past year, future suspect logic on undocumented chronic conditions, alternate submission of ICD-10's, suspect diagnosis, cluster coding analysis, physician reporting metrics, benchmark adjustments for Accountable Care Organizations, revenues for Medicare Advantage and Managed Medicaid, intervention opportunities for care, and HCC weighting report at member level. Those skilled in the art will recognize that other reports can also be generated. The host server 104 transmits the report 138, in the form of a web page 140 as shown in FIG. 5, to the client device 102, where it is displayed on the display 110.

Once the procedure and diagnosis codes are determined, the medical biller will transmit the claim to the payor, such as an insurance company or CMS. This is usually done electronically by using Electronic Data Interchange to submit the claim file to the payer directly or via a clearinghouse. Currently, the ICD codes for identifying the healthcare status of the individual are limited to contracts with Center for Medicare and Medicaid (CMS) services. However, ICD could be used by other payors in conjunction with the present invention.

The payor processes the claims usually through automatic electronic processing. For higher dollar amount claims, the insurance company's medical claims examiners, medical claims adjusters or medical directors review the claims and evaluate their validity for payment using rubrics (procedure) for patient eligibility, Provider credentials, and medical necessity. Approved claims are reimbursed normally at a pre-negotiated between the health care Provider and the insurance company. Failed claims are rejected and notice is sent to Provider.

In an alternate embodiment, the CMS subcontractor provides a pre-determined portion of the CMS margin to an administrator, functioning as a Medical Home Model (generally primary care physician(s) are the Providers of care), of the coding program 122 and reporting program 123. Medical Home—the risk is borne by the insurance company, not the Provider. In this way, the reporting program 123 maximizes the efficiency of the physician to practice holistic versus episodic medicine.

Alternate embodiments of the invention may include applications other than described above, including, but not limited to, Employers who self-funded insurance (under ERISA Laws); Disease Management Case Management Utilization Management programs; Regulatory Compliance; Whistleblower expert witness testimony linked to Medicare Fraud; Provider process improvement training; Revenue cycle and process improvement consulting.

Changes can be made in the above constructions without departing from the scope of the disclosure, it is intended that all matter contained in the above description or shown in the accompanying drawings shall be interpreted as illustrative and not in a limiting sense.

What is claimed is:

1. A method of administering a healthcare analytics process through a computer system having at least one server, at least one client device, and a communication network operatively and electrically connecting the client device to the at least one server, the method comprising the steps of:
providing a coding program running on the at least one server without transmitting advertisements to the at least one client device;
accessing, by a user from the client device, the coding program, and entering search data into the at least one client device by the user;
transmitting the search data to the at least one server; and
generating with the coding program on the at least one server, search results associated with the search data and displaying the search results associated with the search data at the at least one client device.

2. The method of claim 1, further comprising, stitching the search data on the at least one server.

3. The method of claim 1, further comprising, removing duplicates and fixing errors in the search data on the at least one server.
